(19) United States
(12) Patent Application Publication
(10) Pub. No.: US 2019/0240018 A1
(43) Pub. Date: Aug. 8, 2019

(54) PROSTHETIC MITRAL VALVE COMPRISING AN ANNULAR-VENTRICULAR COUPLING MECHANISM

(71) Applicant: The Methodist Hospital, Houston, TX (US)

(72) Inventors: Matthew Scott Jackson, Houston, TX (US); Stephen Rees Igo, League City, TX (US); Stephen H. Little, Houston, TX (US)

(21) Appl. No.: 16/313,771

(22) PCT Filed: Jul. 6, 2017
(86) PCT No.: PCT/US17/40928

§ 371 Date: Dec. 27, 2018

Related U.S. Application Data
(60) Provisional application No. 62/358,636, filed on Jul. 6, 2016.

(51) Int. Cl. A61F 2/24

(52) U.S. Cl. CPC A61F 2/2445; A61F 2/2418; A61F 2220/0008; A61F 2220/0075; A61F 2210/0014

(57) ABSTRACT

A prosthetic mitral valve comprising a flexible frame comprising an annular base and a pair of diametrically-opposed struts extending downwardly from the annular base; a sewing ring mounted to the annular base for securing the prosthetic mitral valve in an annulus vacated by a native mitral valve; a pair of leaflets mounted to the annular base and the pair of diametrically-opposed struts; and a pair of coupling sutures configured to extend from the sewing ring, down the pair of diametrically-opposed struts, and across a left ventricle for securing the prosthetic mitral valve to papillary muscles.

### REFERENCE TO PENDING PRIOR PATENT APPLICATION

This patent application claims benefit of pending prior U.S. Provisional Patent Application Ser. No. 62/358,636, filed Jul. 6, 2016 by The Methodist Hospital and Matthew Scott Jackson et al. for PROSTHETIC MITRAL VALVE COMPRISING AN ANNULAR-VENTRICULAR COUPLING DEVICE (Attorney's Docket No. METHODIST-20 PROV), which patent application is hereby incorporated herein by reference.

### FIELD OF THE INVENTION

This invention relates to surgical methods and apparatus in general, and more particularly to surgical methods and apparatus for improving mitral valve function.

### BACKGROUND OF THE INVENTION

The human heart consists of four chambers (the right atrium, the right ventricle, the left atrium and the left ventricle) and four valves (the tricuspid valve located between the right atrium and the right ventricle, the pulmonary valve located at the exit of the right ventricle, the mitral valve located between the left atrium and the left ventricle, and the aortic valve located at the exit of the left ventricle). See FIG. 1. The right atrium receives deoxygenated blood from the body, the right ventricle pumps deoxygenated blood to the lungs for oxygenation, the left atrium receives oxygenated blood from the lungs, and the left ventricle pumps oxygenated blood to the body.

As noted above, the mitral valve is located between the left atrium and the left ventricle. A properly functioning mitral valve permits blood to flow from the left atrium to the left ventricle when the left ventricle expands (i.e., during diastole), and prevents the regurgitation of blood from the left ventricle back into the left atrium when the left ventricle contracts (i.e., during systole).

The mitral valve is generally characterized by an annulus which is attached to surrounding tissue, leaflets which open and close during valve function, and chordae tendineae which connect the leaflets to papillary muscles that extend from the lower ventricular wall. See FIG. 2, which shows the mitral valve in an open position as the left ventricle expands (i.e., during diastole); and FIG. 3, which shows the mitral valve in a closed position as the left ventricle contracts (i.e., during systole).

In some circumstances the mitral valve may fail to function properly, such that regurgitation may occur. Such regurgitation reduces the heart's pumping efficiency. By way of example, mitral regurgitation is a common occurrence in patients with heart failure. Mitral regurgitation in patients with heart failure is typically caused by changes in the geometric configurations of the left ventricle, papillary muscles and mitral annulus. These anatomical changes frequently result in incomplete coaptation of the mitral leaflets during systole, resulting in mitral regurgitation.

Where mitral regurgitation is caused by changes in the geometric configurations of the left ventricle, papillary muscles and mitral annulus, the mitral regurgitation is commonly treated by plicating the mitral valve annulus so as to correct the shape of the distended annulus and restore the original geometry of the mitral valve annulus. More particularly, current surgical practice for mitral valve repair generally requires that the distended mitral valve annulus be restored by surgically opening the heart and then fixing sutures, or more commonly sutures in combination with a support ring, to the internal surface of the annulus; this structure is then used to draw the annulus, in a pursestring-like fashion, back into its proper configuration, thereby improving leaflet coaptation and reducing mitral regurgitation. This method of mitral valve repair, generally referred to as “annuloplasty”, effectively reduces mitral regurgitation in heart failure patients. This, in turn, reduces the symptoms associated with heart failure, improves the patient's quality of life and increases patient longevity.

Unfortunately, however, such mitral valve surgery is not effective in all situations, and in some circumstances it may be necessary to replace the natural mitral valve with a prosthetic mitral valve. In this situation, the prosthetic mitral valve typically comprises a rigid annulus sized to be received in the seat of the native mitral valve, and a plurality of leaflets mounted to the rigid annulus. The rigid annulus includes a sewing ring so that the prosthetic mitral valve can be sewn into position at the seat of the native mitral valve. However, the prosthetic mitral valve typically does not have its rigid annulus or leaflets anchored to the papillary muscles of the left ventricle, and hence does not accurately mimic the action of the native mitral valve (which has its leaflets anchored to the papillary muscles by chordae tendineae). More particularly, the natural mitral annulus is flexible with anterior-to-posterior motion. The natural annular-ventricular coupling of the mitral valve leaflets to the papillary muscles (via the chordae tendineae) prevents ventricular dilation, preserves heart size and shape, and maintains cardiac function.

Thus there is a need for a new and improved prosthetic mitral valve which more accurately mimics the action of the native mitral valve.

### SUMMARY OF THE INVENTION

The present invention comprises the provision and use of a new and improved prosthetic mitral valve which more accurately mimics the action of the native mitral valve. More particularly, the new and improved prosthetic mitral valve comprises a single flexible frame comprising an annular base and a pair of diametrically-opposed struts extending downwardly from the annular base. A sewing ring is mounted to the annular base so that the annular base can be secured in the seat of the native mitral valve. Two leaflets are mounted to the annular base and the pair of diametrically-opposed struts. A pair of coupling sutures extend from the sewing ring, down the pair of diametrically-opposed struts, and across the left ventricle to the papillary muscles. In this way, by connecting the new and improved prosthetic mitral valve to both the annulus of the native mitral valve and the papillary muscles, the prosthetic mitral valve more accurately mimics the action of the native mitral valve.

In one preferred form of the invention, there is provided a prosthetic mitral valve comprising:

a flexible frame comprising an annular base and a pair of diametrically-opposed struts extending downwardly from the annular base;

a sewing ring mounted to the annular base for securing the prosthetic mitral valve in an annulus vacated by a native mitral valve;

a pair of leaflets mounted to the annular base and the pair of diametrically-opposed struts; and

a pair of coupling sutures configured to extend from the sewing ring, down the pair of diametrically-opposed struts, and across a left ventricle for securing the prosthetic mitral valve to papillary muscles.

In another preferred form of the invention, there is provided a method for improving mitral valve function, the method comprising:

providing a prosthetic mitral valve comprising:
a flexible frame comprising an annular base and a pair of diametrically-opposed struts extending downwardly from the annular base;
a sewing ring mounted to the annular base for securing the prosthetic mitral valve in an annulus vacated by a native mitral valve;
a pair of leaflets mounted to the annular base and the pair of diametrically-opposed struts; and
a pair of coupling sutures configured to extend from the sewing ring, down the pair of diametrically-opposed struts, and across a left ventricle for securing the prosthetic mitral valve to papillary muscles;

removing a native mitral valve;

securing the pair of coupling sutures to the papillary muscles;

securing the sewing ring at the seat of the native mitral valve;

tensioning the pair of coupling sutures so as to set the tension between the annular base and the papillary muscles; and

securing the pair of coupling sutures to the sewing ring.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view of the human heart;

FIGS. 2 and 3 are schematic views of the mitral valve;

FIGS. 4-6 are schematic views showing a new and improved prosthetic mitral valve formed in accordance with the present invention;

FIGS. 7-20 are schematic views showing further construction details of the new and improved prosthetic mitral valve of FIGS. 4-6;

FIG. 21A is a bottom view of the prosthetic mitral valve of FIGS. 4-6 showing the leaflets attached to the sewing ring and the pair of diametrically-opposed struts, with the anterior and posterior leaflets shown in a neutral configuration;

FIG. 21B is a side view of the prosthetic mitral valve of FIGS. 4-6 showing the arch of the anterior portion of the annular base;

FIG. 21C is a bottom view of the prosthetic mitral valve of FIGS. 4-6, with the anterior and posterior leaflets shown in an open configuration, similarly to diastole;

FIGS. 21D and 21E are in vitro images of the leaflets of the prosthetic mitral valve of FIGS. 4-6 shown in a closed configuration (systolic coaptation), taken from the view of the apex and the left atrium, respectively;

FIGS. 21F and 21G are in vitro images of the anterior and posterior leaflets of the prosthetic mitral valve of FIGS. 4-6 shown in an open configuration (diastolic orifice), taken from the view of the apex and the left atrium, respectively; and

FIGS. 22, 23, 24A-24D, 25A-25C and 26-29 are schematic views showing the new and improved prosthetic mitral valve of FIGS. 4-6 implanted in the heart of a patient.

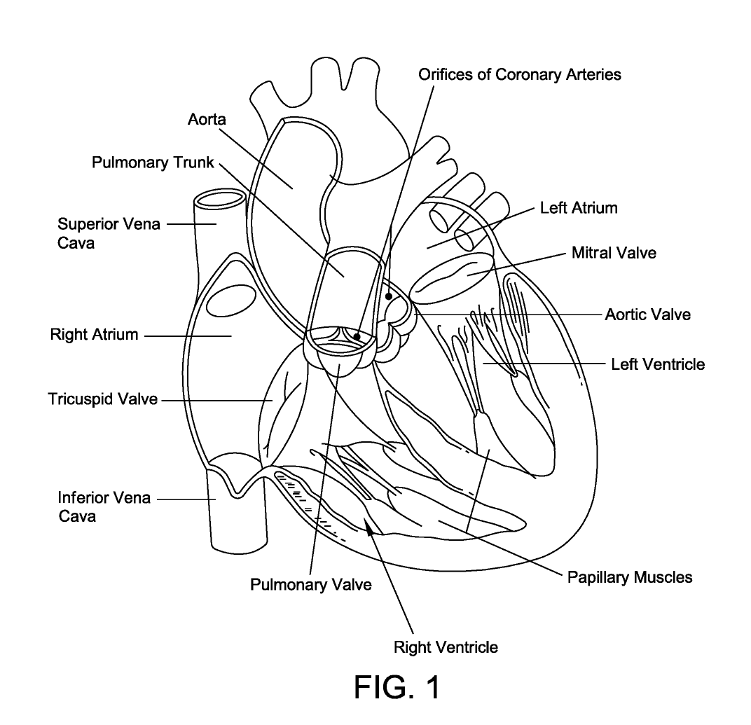

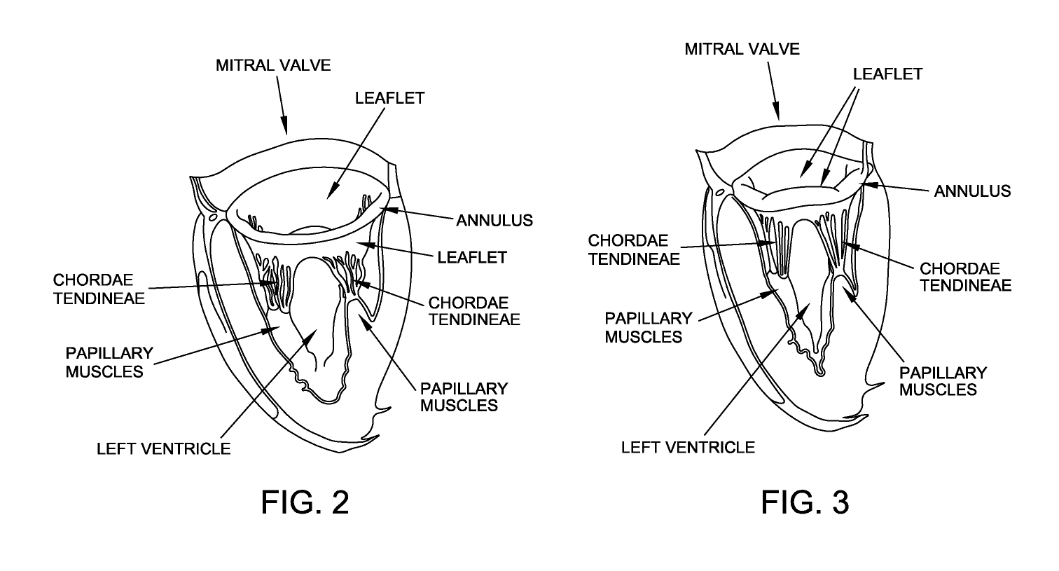

### DETAILED DESCRIPTION OF THE PREFERRED EMBODIMENTS

The present invention comprises the provision and use of a new and improved prosthetic mitral valve 5 which more accurately mimics the action of the native mitral valve.

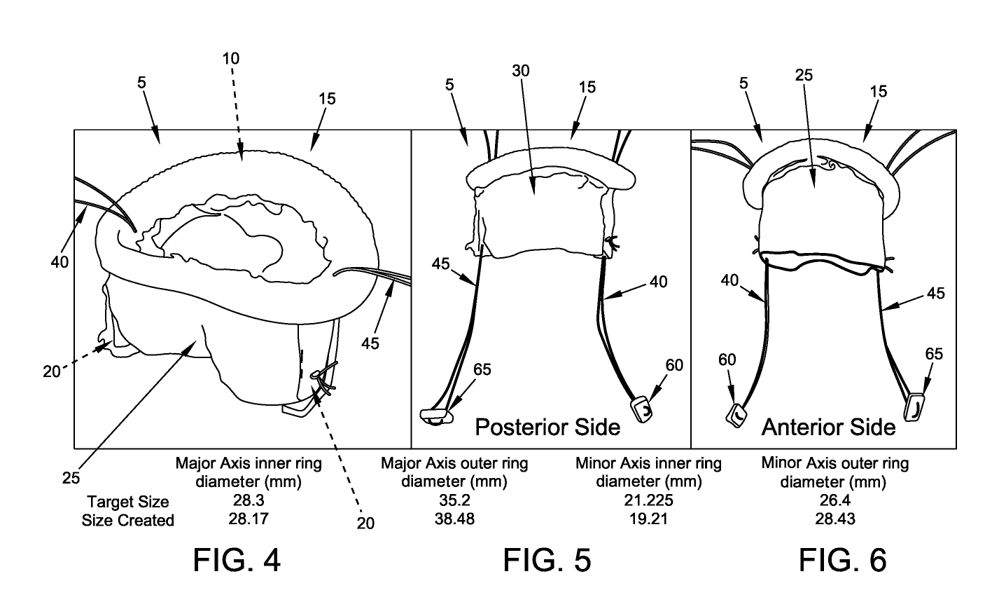

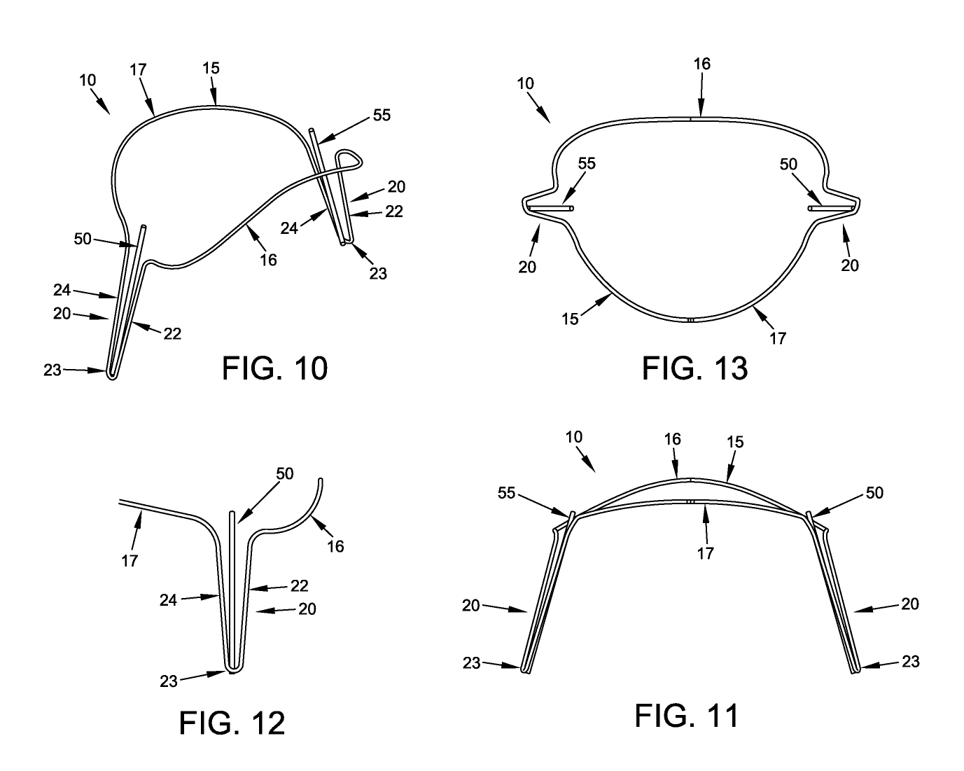

More particularly, and looking now at FIGS. 4-13, the new and improved prosthetic mitral valve 5 comprises a single flexible frame 10 comprising an annular base 15 and a pair of diametrically-opposed struts 20 extending downwardly from annular base 15. An anterior leaflet 25 and a posterior leaflet 30 are mounted to annular base 15 and the pair of diametrically-opposed struts 20. A sewing ring 35 is mounted to annular base 15 so that the annular base can be secured in the seat of the native mitral valve. A pair of coupling sutures extend from sewing ring 35, down the pair of diametrically-opposed struts 20, and across the left ventricle in order to secure prosthetic mitral valve 5 to the papillary muscles. In this way, by connecting the new and improved prosthetic mitral valve to both the annulus of the native mitral valve and the papillary muscles, the prosthetic mitral valve more accurately mimics the action of the native mitral valve.

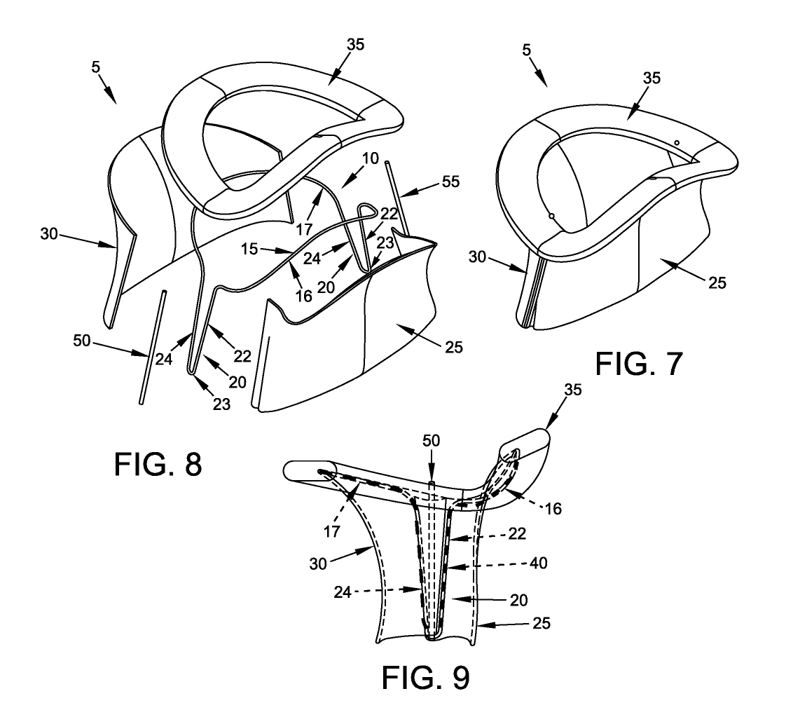

As stated above, and shown in more detail in FIGS. 7-13, prosthetic mitral valve 5 comprises a flexible frame 10 comprising an annular base 15 and a pair of diametrically-opposed struts 20 extending downwardly from annular base 15.

Annular base 15 comprises an anterior portion 16 and a posterior portion 17. Each one of the pair of diametrically-opposed struts comprises a first leg 22 which is connected to anterior portion 16 at the top end of first leg 22 and a second leg 24 which is connected to posterior portion 17 at the top end of second leg 24. The lower ends of first leg 22 and second leg 24 are connected together at a hinge point 23.

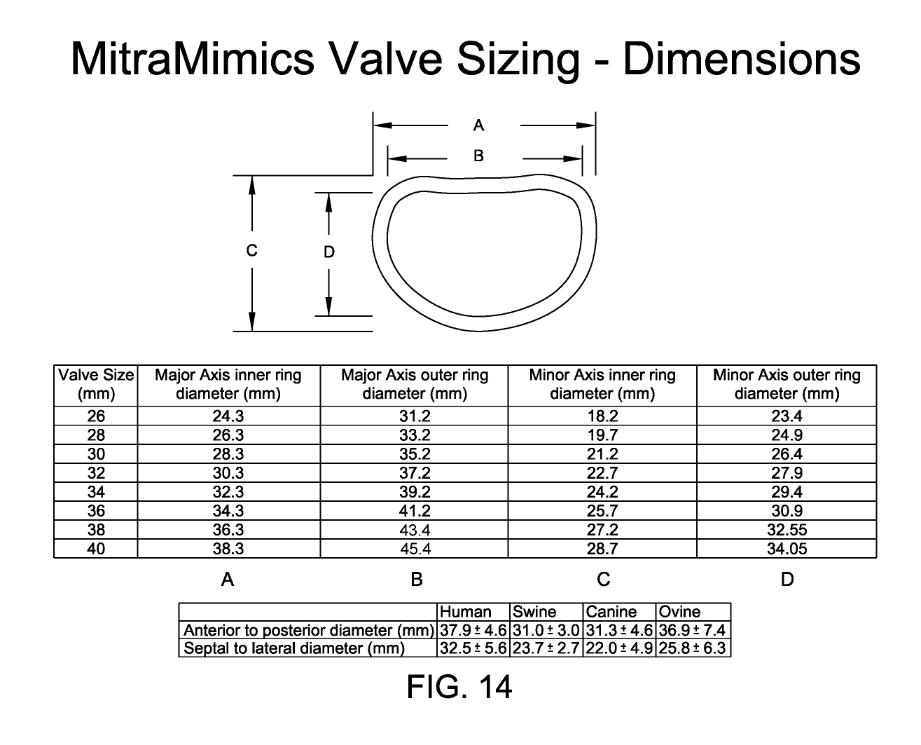

In one preferred form of the present invention, annular base 15 and the pair of diametrically-opposed struts 20 are formed into the saddle shape shown in FIG. 8 from a single length of wire, which is preferably a highly elastic wire such as a shape memory alloy wire (e.g., Nitinol). With such a construction, annular base 15 and diametrically-opposed struts 20 of flexible frame 10 can flex as needed as the surrounding heart anatomy cycles from diastole to systole, and from systole to diastole. Among other things, with the present invention, anterior portion 16 and posterior portion 17 of annular base 15 can flex relative to one another during heart cycling, whereby to better conform to the surrounding anatomy. In this respect it will be appreciated that this flexing of annular base 15 is enhanced by virtue of the fact that anterior portion 16 and posterior portion 17 of annular base 15 are connected to one another through diametrically-opposed struts 20, which act as something of a hinge mechanism between anterior portion 16 and posterior portion 17 of annular base 15. Furthermore, with the present invention, the lower ends (i.e., hinge points 23) of diametrically-opposed struts 20 are free to move toward and away from one another during heart cycling, whereby to better conform to the surrounding anatomy. It will be appreciated that the various elements of flexible frame 10 are sized to mimic the native mitral valve. See for example, FIG. 14, which provides illustrative sizing dimensions.

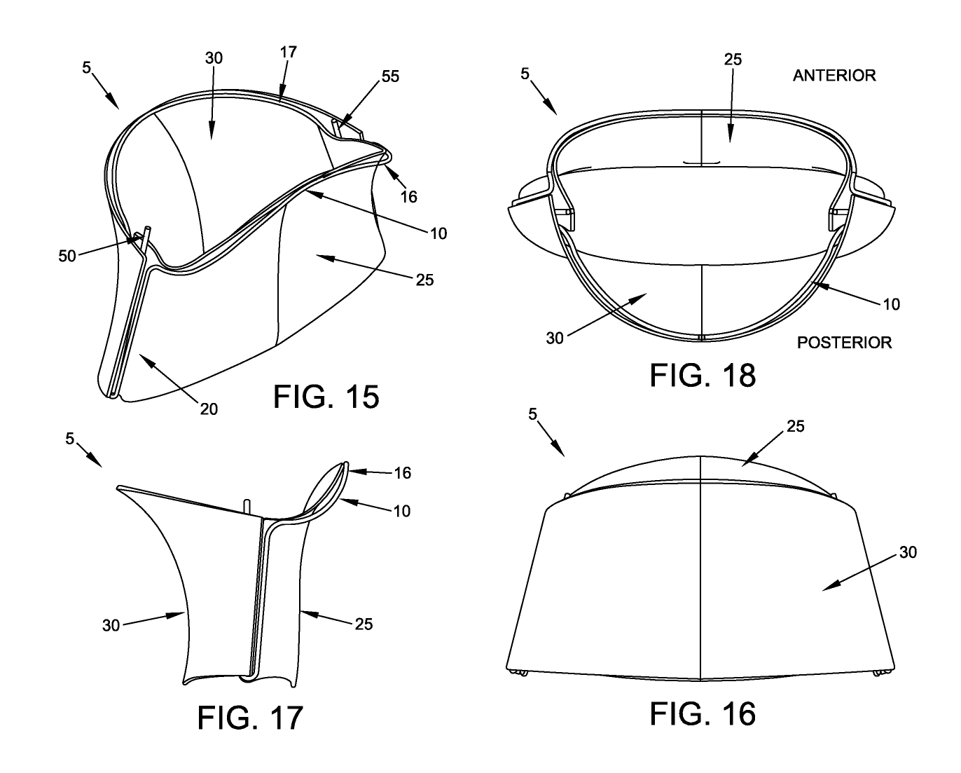

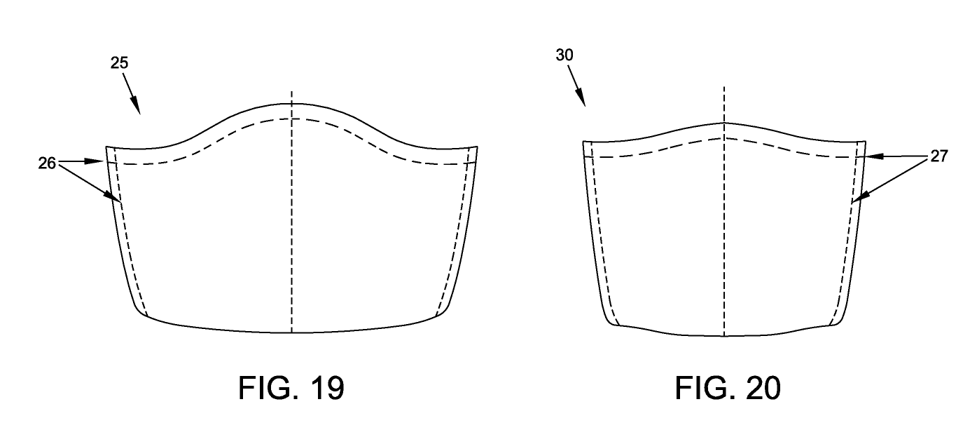

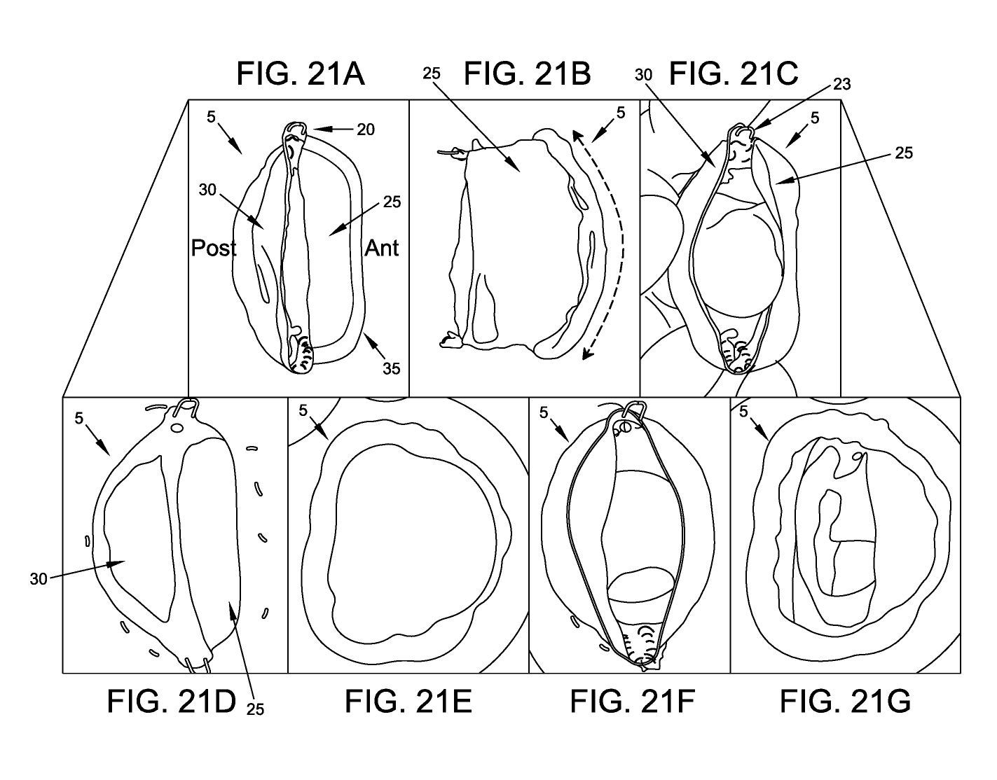

Looking now at FIGS. 4-9 and 15-20, anterior leaflet 25 and posterior leaflet 30 are mounted to annular base 15 and the pair of diametrically-opposed struts 20. In one preferred form of the invention, anterior leaflet 25 is sutured to anterior portion 16 of annular base 15 and to the pair of diametrically-opposed struts 20 along suture line 26 (FIG. 19), and posterior leaflet 30 is sutured to posterior portion 17 of annular base 15 and to the pair of diametrically-opposed struts 20 along suture line 27 (FIG. 20). Each leaflet follows the curvature of flexible frame 10 at the connection point of the leaflet to annular base 15 and the pair of diametrically-opposed struts 20. In this way, and as shown in FIGS. 21A-21G, leaflets 25, 30 are able to open during diastole and close during systole. Note that in one preferred form of the invention, leaflets 25, 30 are asymmetrical, in order that they may mimic the ejection flow of the native mitral valve, i.e., anterior leaflet 25 preferably creates a concave tunnel, whereby to provide an optimal ejection outflow.

In another form of the present invention, the two valve leaflets could be formed from a tube rather than from two pieces.

As stated above, and shown in FIGS. 4-9, suture or sewing ring 35 is mounted to annular base 15 so that the annular base can be secured in the seat of the native mitral valve.

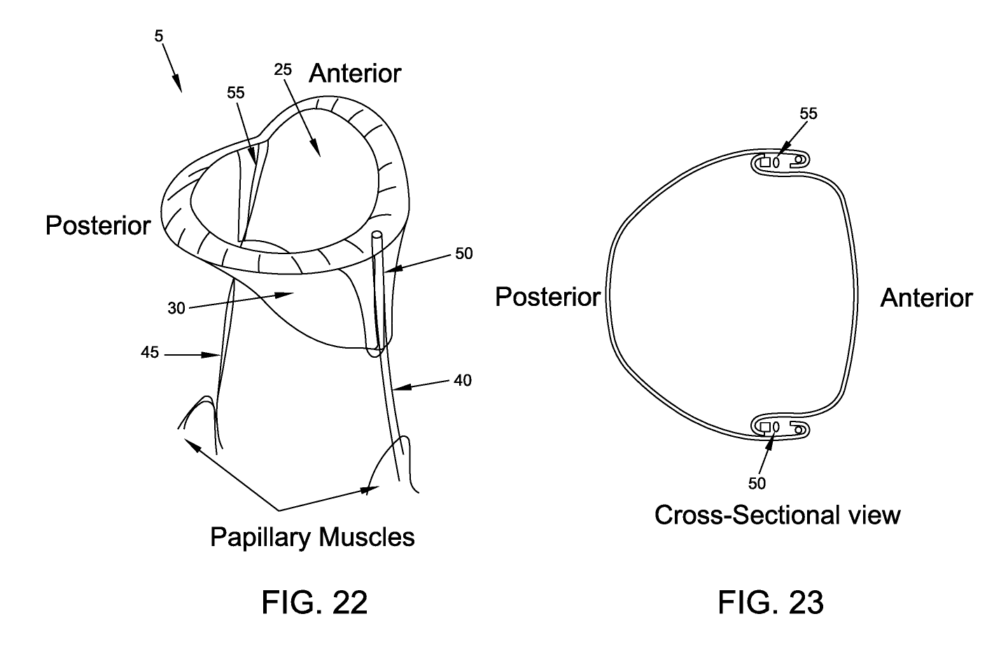

A pair of coupling sutures 40, 45 (FIGS. 4-6) extend from sewing ring 35, down the pair of diametrically-opposed struts 20 and across the left ventricle to the papillary muscles. See FIGS. 22 and 23. Thus, with the present invention, the chordae tendineae of the native mitral valve are “retained” by the provision of coupling sutures 40, 45, whereby to provide superior valve function and help prevent “ballooning” of the left ventricle. Note, however, that with new and improved prosthetic mitral valve 5, the “chordae tendineae” (i.e., coupling sutures 40, 45) extend between annular base 15 of flexible frame 10 and the papillary muscles, and are not secured directly to the two leaflets 25, 30. If desired, suture channels 50, 55 (e.g., tubular structures) may be provided alongside each of the diametrically-opposed struts 20 to coupling sutures 40, 45 through the assembly. Furthermore, if desired, coupling sutures 40, 45 may be passed along some or all of the perimeter of flexible frame 10, e.g., in the manner shown in FIG. 9. In order to assist the surgeon in securing coupling sutures 40, 45 to the papillary muscles, pledgets 60, 65 may be mounted on the lower ends of coupling sutures 40, 45. As will be discussed in more detail below, coupling sutures 40, 45 may be tensioned and then fixed in length, e.g., such as with a knot.

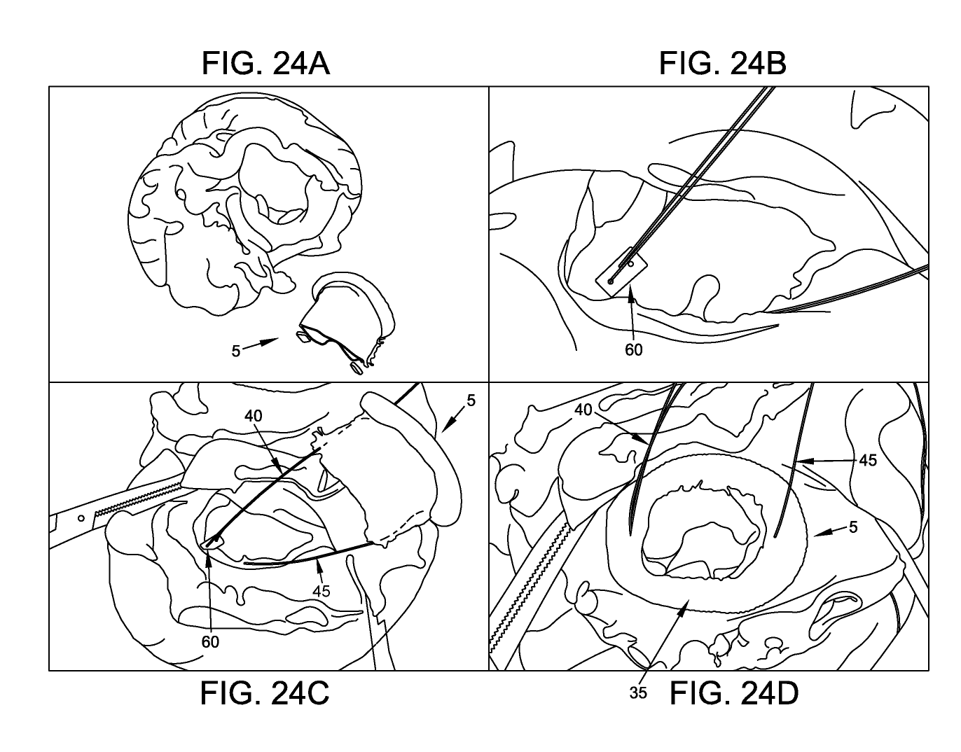

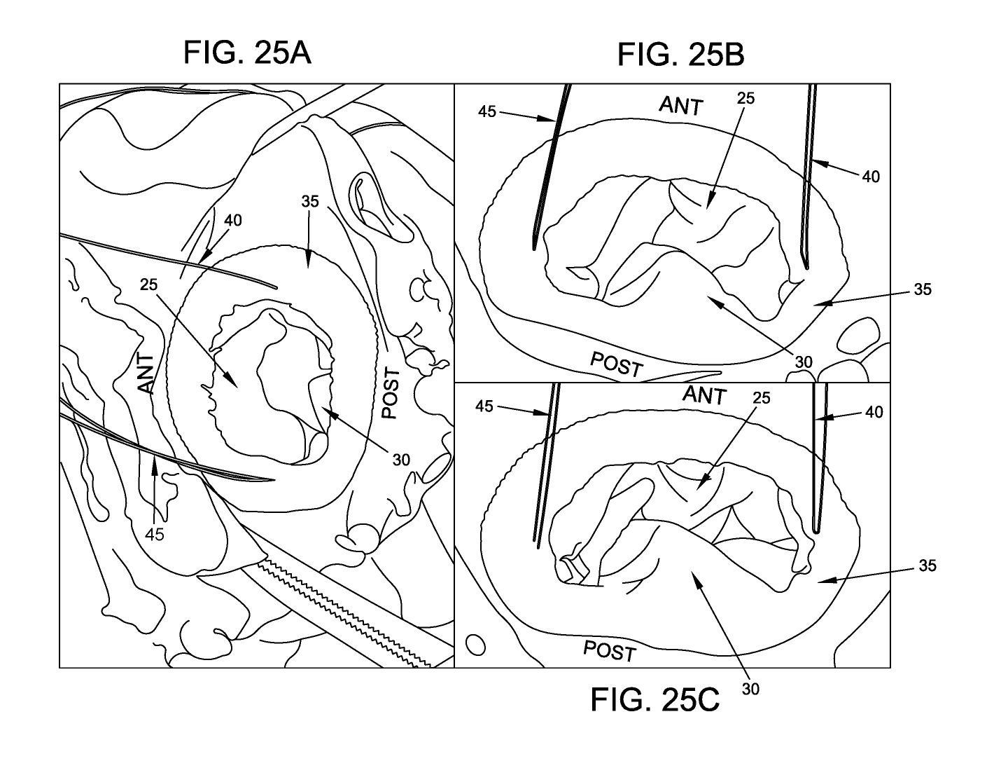

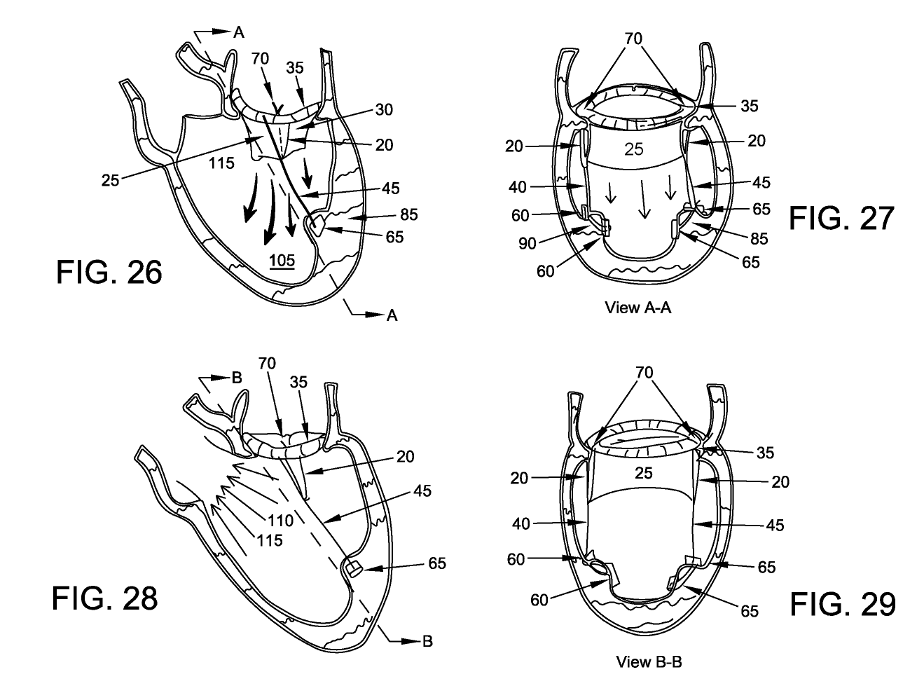

In use, and looking now at FIGS. 24A-24D, 25A-25C and 26-29, the surgeon removes the native mitral valve, secures the lower ends of coupling sutures 40, 45 to the papillary muscles (e.g., with pledgets 60, 65), positions sewing ring 35 at the seat of the native mitral valve, and sutures sewing ring 35 to the seat of the native mitral valve. Then the surgeon tensions the upper ends of coupling sutures 40, 45 so as to set the tension between annular base 15 of prosthetic mitral valve 5 and the papillary muscles. Preferably tensioning is effected by filling the left ventricle with saline while the leaflets 25, 30 are closed, which allows dynamic tensioning of coupling sutures 40, 45 when the left ventricle is fully expanded (i.e., during diastole). Once the tension between annular base 15 and the papillary muscles has been appropriately set, coupling sutures 40, 45 are secured to sewing ring 35, e.g., by tying the coupling sutures with a knot 70 flush with sewing ring 35.

It will be appreciated that by connecting the prosthetic mitral valve to both the seat of the native mitral valve (i.e., by sewing ring 35) and the papillary muscles (i.e., by coupling sutures 40, 45), the prosthetic mitral valve more accurately mimics the action of the native mitral valve. The new and improved prosthetic mitral valve includes a saddle-shaped, physiologically dynamic flexible annulus (annular base 15 and sewing ring 35); ventricular-annular coupling from annular base 15 and sewing ring 35 and papillary muscles 85 and 90 (via coupling sutures 40, 45); and a non-symmetric posterior and anterior valve leaflet design (e.g., a non-symmetric posterior valve leaflet 30 and an anterior valve leaflet 25). Ventricular flow streamlines during diastole 105 (i.e., when the left ventricle expands and blood flows from the left atrium to the left ventricle), and systole 110 (i.e., when the left ventricle contracts and blood is prevented from regurgitating from the left ventricle back into the left atrium), demonstrate efficient, physiologic hemodynamics without obstruction of the left ventricular outflow tract 115.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A prosthetic mitral valve comprising:
a flexible frame comprising an annular base and a pair of diametrically-opposed struts extending downwardly from the annular base;
a sewing ring mounted to the annular base for securing the prosthetic mitral valve in an annulus vacated by a native mitral valve;
a pair of leaflets mounted to the annular base and the pair of diametrically-opposed struts; and
a pair of coupling sutures configured to extend from the sewing ring, down the pair of diametrically-opposed struts, and across a left ventricle for securing the prosthetic mitral valve to papillary muscles.

2. A prosthetic mitral valve according to claim 1 wherein the annular base comprises an anterior portion and a posterior portion.

3. A prosthetic mitral valve according to claim 1 wherein each of the pair of diametrically-opposed struts comprises a first leg and a second leg connected together at a hinge point.

4. A prosthetic mitral valve according to claim 3 wherein the first leg of each of the pair of diametrically-opposed struts is mounted to the anterior portion of the annular base, and the second leg of each of the pair of diametrically-opposed struts is mounted to the posterior portion of the annular base.

5. A prosthetic mitral valve according to claim 1 wherein the frame comprises a shape memory alloy wire.

6. A prosthetic mitral valve according to claim 1 wherein the pair of leaflets are mounted to the annular base and the pair of diametrically-opposed struts by a suture.

7. A prosthetic mitral valve according to claim 1 wherein the pair of leaflets are asymmetrical.

8. A prosthetic mitral valve according to claim 1 further comprising a pair of suture channels mounted to the flexible frame for receiving the pair of coupling sutures.

9. A prosthetic mitral valve according to claim 1 further comprising a pair of pledgets for securing the pair of coupling sutures to the papillary muscles.

10. A method for improving mitral valve function, the method comprising:
providing a prosthetic mitral valve comprising:
a flexible frame comprising an annular base and a pair of diametrically-opposed struts extending downwardly from the annular base;
a sewing ring mounted to the annular base for securing the prosthetic mitral valve in an annulus vacated by a native mitral valve;
a pair of leaflets mounted to the annular base and the pair of diametrically-opposed struts; and
a pair of coupling sutures configured to extend from the sewing ring, down the pair of diametrically-opposed struts, and across a left ventricle for securing the prosthetic mitral valve to papillary muscles;
removing a native mitral valve;
securing the pair of coupling sutures to the papillary muscles;
securing the sewing ring at the seat of the native mitral valve;
tensioning the pair of coupling sutures so as to set the tension between the annular base and the papillary muscles; and
securing the pair of coupling sutures to the sewing ring.

11. A method according to claim 10 wherein pledgets are used to secure the pair of coupling sutures to the papillary muscles.

12. A method according to claim 10 wherein sutures are used to secure the sewing ring to the seat of the native mitral valve.

13. A method according to claim 10 wherein tensioning is effected by filling the left ventricle with saline while the pair of leaflets are closed.

14. A method according to claim 10 wherein the coupling sutures are secured to the sewing ring by tying the sutures in a knot.

15. A method according to claim 10 wherein the annular base comprises an anterior portion and a posterior portion.

16. A method according to claim 10 wherein each of the pair of diametrically-opposed struts comprises a first leg and a second leg connected together at a hinge point.

17. A method according to claim 16 wherein the first leg of each of the pair of diametrically-opposed struts is mounted to the anterior portion of the annular base, and the second leg of each of the pair of diametrically-opposed struts is mounted to the posterior portion of the annular base.

18. A method according to claim 10 wherein the frame comprises a shape memory alloy wire.

19. A method according to claim 10 wherein the pair of leaflets are mounted to the annular base and the pair of diametrically-opposed struts by a suture.

20. A method according to claim 10 wherein the pair of leaflets are asymmetrical.

21. A method according to claim 10 further comprising a pair of suture channels mounted to the flexible frame for receiving the pair of coupling sutures.
